(19) United States
(12) Patent Application Publication
(10) Pub. No.: US 2021/0338872 A1
(43) Pub. Date: Nov. 4, 2021

(54) Disinfecting Fogger

(71) Applicant: Brian Krawczyk, Zion, IL (US)

(72) Inventor: Brian Krawczyk, Zion, IL (US)

(21) Appl. No.: 17/031,994

(22) Filed: Sep. 25, 2020

Related U.S. Application Data
(60) Provisional application No. 63/018,726, filed on May 1, 2020.

(51) Int. Cl. A61L 2/22; A61L 2/26

(52) U.S. Cl. CPC A61L 2/22; A61L 2101/18; A61L 2/26

(57) ABSTRACT

This present invention relates to a novel disinfectant fogger device and a disinfecting solution contained therein for disinfecting or sanitizing an enclosed space. The disinfecting solution is intended to kill germs, bacteria, viruses and other harmful microbes. The disinfectant fogger device is comprised of an aerosol container, the novel disinfecting solution, and a nozzle for dispensing the solution on demand. The disinfectant solution is preferably comprised of between 5-15% of a quaternary ammonium compound by weight, between 40-70% of a monohydric alcohol by weight, and between 15-40% of an alkyl by weight, and is capable of killing up to 99.9% of the germs and other harmful pathogens in the enclosed space.

### CROSS-REFERENCE TO RELATED APPLICATION

The present application claims priority to, and the benefit of, U.S. Provisional Application No. 63/018,726, which was filed on May 1, 2020 and is incorporated herein by reference in its entirety.

### BACKGROUND

The present invention relates generally to the field of disinfecting or sanitizing devices. More specifically, the present invention relates to a disinfecting fogger device for releasing a disinfectant for eliminating airborne bacteria, viruses, germs, fungi and the like within a confined or semi-confined area such as a room in a home, hospital, restaurant, funeral parlor, classroom, office, conference room, motel room, etc. More specifically, the disinfecting or sanitizing fogger device of the present invention is a spray or mist emitting device that offers complete area coverage for disinfecting a room or enclosed space, and eliminating substantially all of the germs, bacteria, viruses, etc. The disinfecting fogger device of the present invention comprises a pressure-activated cylinder that contains a disinfectant solution or material, and a nozzle disinfectant spray opening that, upon activation, releases the pressurized disinfectant solution throughout an entire room or area to be treated. To activate, the nozzle is pushed downwards using a flap present on the nozzle head so that the pressurized disinfectant may escape through the opening in the nozzle. As the disinfectant leaves the nozzle spray opening, it is released in an upward and outward direction from the canister to disinfect the entire room and enclosed space at the same time, and the mist particles are permitted to fall via gravity onto any surfaces in the room. The disinfectant solution used in the device of the present invention includes a mixture of approximately 12% of ammonium saccharinate, 56% of ethanol, and 32% of an alkyl (all percentages are by weight). This particular mixture of disinfectant solution kills up to 99.9% of airborne, germs, pathogens, viruses, and the like. Accordingly, the present specification makes specific reference thereto. However, it is to be appreciated that aspects of the present invention are also equally applicable to other like applications, devices and methods of manufacture.

By way of background, bacterial, fungal, viral and microbial contamination of rooms, offices, restaurants, schools and other confined spaces has long been a source of infection and disease for humans. Bacteria, viruses, and other disease-causing microbes adhere to surfaces after contact with humans, animals, foods, etc., and also linger in the air within a room after being discharged by a person sneezing, coughing, laughing and even speaking. In this manner, humans have spread infectious diseases among each other since time immemorial including, without limitation, the common cold, influenza, rotavirus, hepatitis A, tuberculosis, conjunctivitis, staphylococcal bacterial infections, strep throat and other streptococcal bacterial infections, as well as the ongoing Covid-19 pandemic. Accordingly, eliminating, reducing, and diminishing bacteria, viruses and other harmful microbes is a major concern for all individuals as well as for the owners and operators of hotels, restaurants, arenas and other public gathering places. Bacteria and viruses may cause sicknesses, such as a cold or flu, viral outbreaks, and irritation or exasperation of existing allergies, asthma and other breathing related issues, if not properly addressed.

In fact, according to the Centers for Disease Control and Prevention (CDC), approximately two million people become sick due to infections caused by antibiotic-resistant bacteria each year. At least 23,000 people die as a direct result of these infections. Many individuals who officially succumb to other conditions do so after their health is compromised by an antibiotic-resistant infection. Overuse of antibiotics is a major factor in the increase in antibiotic resistance, and it's a problem everyone can help solve by taking steps to eliminate germs and other infectious agents before they get the chance to cause illness.

In many cases, to maintain a properly sterilized and sanitized environment, manual labor is typically required by individuals and may be supplemented by robotic devices. For example, individuals spend a considerable amount of time cleaning and disinfecting a home, office, school, businesses, public spaces or other areas. Manually cleaning every surface is time consuming, tiring, ineffective, and may result in some spots or areas being left unclean and untreated. This can lead to the spread of germs, bacteria, and dangerous viruses. Stated differently, it is difficult to ensure a high-level disinfection using manual cleaning and sanitizing efforts because of the amount of time and effort involved, the possibility of missed surfaces, re-contamination from dirty sponges, mops, rags and the like, and the improper use and mixing of antibacterial cleaning solutions.

Further, in today's clinical environment, the demand for clean working space is at an all-time premium. More specifically, medical examining rooms, surgical rooms, waiting rooms and patient rooms are very expensive to build and maintain, and most hospitals and clinics seek to utilize these spaces to the maximum possible. With the high turnover rates of these spaces (e.g., after each patient encounter, surgery, etc.) there is a demand on the cleaning staff to prepare each of the rooms and restore them to the required conditions before the next patient is seen for their appointment or procedure. This means that working surfaces, such as trays, examining tables, table tops, beds, etc., must also be cleaned, disinfected and/or sanitized. This can be done by the use of chemicals sprays or towelettes that have been saturated with disinfectant chemistry, or other methods that help restore the area to the required levels of protection for the healthcare professionals, patients and their guests. This effort is labor intensive and can still result in some surfaces in the area of concern being left untreated, given the fact that it is not possible for the staff person to visibly see which surfaces have been cleaned, and which ones have not.

Additionally, airborne bacteria, germs and viruses are oftentimes controlled through the use of sprays, filters, ultraviolet light emitters and/or air cleaning devices. For example, filters within heating, ventilation, and air conditioning (HVAC) systems may include chemical treatments and other electrostatic emitting devices to reduce the concentration of airborne bacteria, germs and viruses in the airflow being processed by such systems. Specially designed filter devices may also be placed on a floor or a table and help to filter and recirculate air being treated through the filter device, or may be part of a central or forced air system that provides heating and cooling for a home, business or other enclosed space. Furthermore, sprays have been developed for use with a handheld aerosol canister and may be sprayed over a limited area for a short period of time, but are typically only useful when treating the surface area in the immediate vicinity of where the user and can are located.

Therefore, there exists a long felt need in the art for a portable and reusable disinfectant product that can be employed within homes, businesses, restaurants, schools, hospitals, and other enclosed areas quickly, effectively and with minimal risk. There is also a long felt need in the art for a disinfectant fogger that emits a disinfecting spray designed to completely disinfect the enclosed space in which it is discharged into, and that effectively eliminates unpleasant odors, bacteria, germs, and viruses from the surroundings. More specifically, there is a long felt need in the art for a disinfectant fogger that allows the spray to permeate an entire room, covering all surfaces and hard to reach areas that may be missed by a user wielding a handheld device, and that enables users to maintain proper sanitary conditions within a specific location with a consistent mist droplet size and dispersal rate. Further, there is a long felt need in the art for a method of using a disinfectant product that requires minimal effort or input from the user, eliminates user error, and is safe for residential use where both people and pets may cohabitate. Finally, there is a long felt need in the art for a disinfectant product that is relatively inexpensive to manufacture, is exceedingly effective, and that is safe and easy to use.

The subject matter disclosed and claimed herein, in one embodiment thereof, comprises a disinfecting fogger device product having a pressure-activated cylinder, a disinfectant material or solution stored in said container, and a nozzle containing a disinfectant spray opening that upon activation, releases the disinfectant solution throughout an entire room or area in a mist like state, wherein the nozzle is activated by pushing downwards towards the cylinder using a flap present on the nozzle to create an opening or pathway for the pressurized contents of the cylinder to flow through in order to release the disinfectant solution into the surrounding atmosphere. Additionally, the disinfecting fogger device of the present invention is also aesthetically pleasing and cost effective, as well as being safe as it releases the disinfectant only upon the activation of the nozzle. The invention also offers a novel and effective disinfectant solution composition that kills 99.9% of germs and is comprised of ammonium saccharinate, ethanol, and alkyl.

In this manner, the disinfectant fogger device of the present invention accomplishes all of the forgoing objectives, and provides a relatively safe, easy, convenient and cost-effective solution to disinfecting an entire enclosed space with a single action. More specifically, the disinfectant fogger device emits a disinfecting solution that is designed to completely disinfect the enclosed space (i.e., even hard to reach spaces and surfaces) in which it is discharged into, and effectively eliminates unpleasant odors, bacteria, germs, and viruses from the surroundings. The disinfectant fogger device also emits a consistent mist droplet size and dispersal rate, and requires minimal effort, input or time commitment from the user, thereby eliminating the possibility of user error.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a disinfecting fogger device comprised of a pressure-activated cylinder, a unique and novel disinfectant solution contained within the cylinder, and a nozzle in fluid communication with the cylinder. The nozzle of the present invention further comprises a disinfectant spray opening that, upon activation of the nozzle's flap, releases the pressurized disinfectant solution throughout an entire room or other enclosed area. More specifically, the nozzle is activated by pushing downwards on the flap (i.e., towards the cylinder). The disinfectant material kills 99.9% of airborne germs, bacteria, viruses and the like by using a unique disinfectant composition that comprises approximately 12% ammonium saccharinate, 56% ethanol, and 32% alkyl (all percentages by weight). The disinfecting fogger device of the present invention is easily portable and has an industrial-strength or heavy duty metal nozzle that does not wear out over time and does not cause inconsistent mist droplet size. The nozzle is optimized for the radial and vertical distribution of the disinfectant solution, and is capable of broadcasting the disinfectant solution onto all surfaces within the room. The nozzle may also include a screen or a mesh to control the droplet size of the disinfectant solution, with the droplet size preferably ranging from about 5 microns to about 50 microns and having a predefined dispensing rate and an effective solution concentration of between 10 to 90%.

In a further embodiment of the present invention, a method of quickly and efficiently disinfecting an entire room or an enclosed space, such as those in a business, home, hospital, school, hotel and the like, to remove bacteria, germs, viruses and microbiological contamination is disclosed. The method comprises the initial step of acquiring the disinfecting fogger device of the present invention and of sufficient capacity to disperse the disinfectant onto substantially all of the surfaces or exposed areas in the room. The next step is to place the disinfecting fogger device substantially in the center of the room or area and at a height that is appropriate for allowing the spray to reach all surfaces. Then, the user activates a nozzle of the fogger device by pressing down the nozzle using a flap present on the nozzle head, and allowing the fogger device to discharge the pressurized disinfectant solution or material contained therein, which has a composition of approximately 12% ammonium saccharinate, 56% ethanol, and 32% alkyl (all percentages based on weight). Next, time is permitted for the expelled disinfectant solution to contact the surfaces in the room, and the room is finally opened again for use once the disinfectant solution has dried.

A further embodiment of the present invention disclosed herein describes a device for dispensing a disinfectant solution into the surrounding area. More specifically, the device is a pressure-activated cylinder that is configured to contain a propellant and a disinfectant solution, and a diffusing nozzle coupled to the cylinder to release the disinfectant solution and the propellant through an opening at the top of the nozzle. The nozzle is present on the top of the cylinder and is activated by pressing the nozzle down towards the cylinder to release the disinfectant solution in a mist like state from the cylinder and in an upward and 360 degree direction. The high pressure of the disinfectant solution is predetermined, and is configured for a conventional space such as a room wherein the fog or mist of the disinfecting solution is capable of killing approximately 99% of pathogens in the air and on the surfaces of the space. As previously stated, the disinfectant solution of the fogger device preferably has a composition of 12% ammonium saccharinate, 56% ethanol, and 32% alkyl (all percentages based on weight). The pressure of most plastic containers generally should not exceed 160 psig, and metal containers up to 200 psig. Accordingly, the pressure is around 140 to 160 psig for applications using the device of the present invention.

Furthermore, in each of the embodiments described herein, the disinfectant solution is optimized for fast or specifically designed drying characteristics, providing dry surfaces within a short time following the activation of the disinfecting fogger device and allowing the room to be returned to use in a quick fashion. A dwell time appropriate for effective surface disinfection is adapted for the specific surfaces to be disinfected. Accordingly, the disinfectant solution may further comprise additional drying elements such as calcium sulfate, sodium sulfate, calcium chloride and magnesium sulfate which can help speed the drying of the disinfectant after application. The drying elements may range in solution from between 0.5 to about 15%, and preferably between 2 and 7%.

In a further embodiment of the presently described invention a disinfecting emitting device is described and includes a rigid container that is sized and configured to receive a disinfecting solution under pressure. The container has a flat base on one end and a nozzle on a second end opposite the flat base. The nozzle is movable between a first and second position and the nozzle being substantially surrounded by a collar. A disinfecting solution is contained within the container and held at a pressure higher than ambient. The disinfecting solution includes between 5-15% of a quaternary ammonium compound, between about 40-70% monohydric alcohol, and between about 15-40% alkyl.

In a yet still further embodiment of the presently described invention, a method of using a disinfecting and sanitizing fogger is described and includes the steps of initially selecting an area to be disinfected and sanitized, and then providing a canister having a unique disinfecting solution. The canister is configured for holding contents under pressure greater than an ambient pressure. The canister has a flat bottom and a nozzle, and of a sensor or timer for use in activation. The nozzle has a first and second position, and the canister includes a screen disposed below the nozzle to provide a droplet size of between 5 and 50 microns. Next, the canister is filled with a solution for disinfecting and sanitizing and is held at a pressure higher than ambient. The disinfecting solution is preferably comprised of between 5-15% of ammonium saccharinate, between about 40-70% monohydric alcohol, and between about 15-40% alkyl (all percentages based on weight). Then, the timer or sensor is set and the nozzle is moved from the first position to the second position to release the disinfectant solution into the area upon a time lapse or sensing event. Once activated, the disinfecting fogger device of the present invention discharges the disinfectant solution in a matter of few seconds, with the spray characteristics optimized for broadcast of the disinfectant solution within the confined space and for quick contact with the targeted surfaces.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the disinfecting fogger spray device of the present invention showing the cylinder and the nozzle components in accordance with the disclosed architecture;

FIG. 2 illustrates a diagrammatic representation of one potential embodiment of the disinfectant solution for use in the disinfecting fogger spray device of the present invention in accordance with the disclosed architecture;

FIG. 3 illustrates a top partial and perspective view of one potential embodiment of the nozzle of the portable disinfectant fogger device of the present invention in an activated state in accordance with the disclosed architecture;

FIG. 4 illustrates a perspective view of one potential embodiment of the disinfectant fogger device of the present invention in an active state in an examining room of a hospital in accordance with the disclosed architecture;

FIG. 5 illustrates a perspective view of one potential embodiment of the disinfectant fogger device of the present invention in an active state in a restaurant area in accordance with the disclosed architecture; and

FIG. 6 illustrates a perspective view of one potential embodiment of the disinfectant fogger device of the present invention in an active state in an office environment in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention or do not limit the scope of the invention. Additionally, an illustrated embodiment need not have all the aspects or advantages shown. Thus, in other embodiments, any of the features described herein from different embodiments may be combined.

As noted above, there is a long felt need in the art for a portable and reusable disinfectant device that can be utilized in an enclosed space quickly, effectively and with minimal risk. There is also a long felt need for a disinfectant fogger device that emits a disinfecting spray designed to completely disinfect the enclosed space into which it is discharged, and that effectively eliminates unpleasant odors, bacteria, germs, and viruses from the surroundings. Further, there is a long felt need in the art for a method of using a disinfectant fogger device that requires minimal effort or input from the user, eliminates or reduces the possibility of user error, and is safe for residential use by non-experts. The disinfectant device of the present invention is not limited to disinfection solutions and can emit sanitizing and antiseptic sprays as well.

The present invention, in one exemplary embodiment, is a portable disinfecting fogger device comprising a pressure-activated cylinder that contains a novel disinfectant solution, and a nozzle on the top of the cylinder that is in fluid communication with the cylinder when activated. More specifically, the nozzle has a disinfectant spray opening that, upon activation of the nozzle, releases the disinfectant colution throughout an entire room or area in an upward and a 360 degree direction. The cylinder contains the pressurized disinfectant solution, which in one embodiment is comprised of a combination of approximately 12% ammonium saccharinate, 56% ethanol, and 32% alkyl. Notwithstanding, other compounds may also be included in the disinfectant solution, such as a drying agent, scent, fragrance and the like. For example, the drying agents may include, but are not limited to, calcium sulfate, sodium sulfate, calcium chloride and magnesium sulfate, which can help speed the drying of the disinfectant after application, such as by absorbing ethanol or other propellants that may be in the solution.

Additionally, a humectant or other hygroscopic substance, such as glycerin, ethylhexylglycerin, dexpanthenol, and a fatty alcohol, alone or in combination, may also be added to the disinfecting solution to ensure the solution has an appropriate surface contact time. Many EPA registered disinfectants have a minimum surface contact time to ensure that the pathogens are effectively destroyed. Disinfecting solutions must typically remain visibly wet for the entire contact time on the surface. Contact times can vary between fifteen seconds to ten minutes depending on the particular disinfecting formula. In embodiments using a humectant, a concentration is selected from a range of approximately 0.5-20 percent based on the desired contact time of the disinfecting solution and the expected surface materials.

Similarly, a surface active compound, such as a surfactant may also be added to the disinfecting solution. A cationic detergent from a group of alkyl- or aryl-substituted quaternary ammonium compounds such as, benzalkonium chloride, benzathonium chloride, cetylpyridinium chloride, each having an ionizable halogen, such as bromide, iodide, or chloride. Quaternary ammonium compounds are effective against most bacteria, enveloped viruses, some fungi (including yeasts), and protozoa. An alternative to a quaternary based compound could be octenidine dihydrochloride for its antisepsis properties.

The disinfecting fogger device of the present invention is easily portable and has an industrial-strength or heavy-duty metal nozzle that does not wear out over time, and does not cause inconsistent mist droplet size. Droplet sizes for use in the present invention that range between 5-50 microns (μm) in diameter have proven to be most effective. Accordingly, the disinfectant solution droplet size is preferably between 10 and 35 microns, which is believed to be ideal to tackle pathogens, vector carriers and other pests. In addition, the active ingredients of the formulation can be applied in concentrations ranging from 10-90%, with the remaining portions making up propellant, stabilizers, drying agents or other non-treatment components, and more preferably from 30-80% and at flow rates of up to 0.52 quarts per minute (31.7 quarts per hour or nearly 8 gallons an hour). The application of the disinfectants, sanitizing solutions and biocides via aerosol or fogging can significantly reduce the number of viable infectious pathogens in a particular area. Foggers produce micro droplets that float in the air for approximately 10 minutes after application, reaching the most inaccessible parts of a room or area where conventional cleaning or spraying typically can't reach. The smaller the droplet, the longer the particles will remain airborne. It is estimated that approximately one ounce of disinfectant solution will typically cover around 1,000 cubic feet.

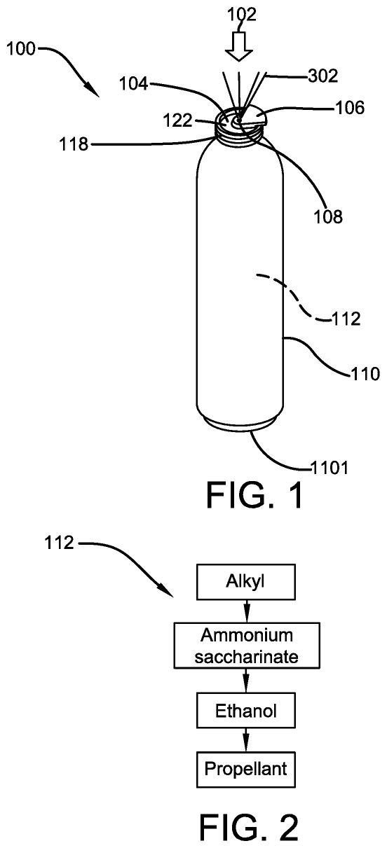

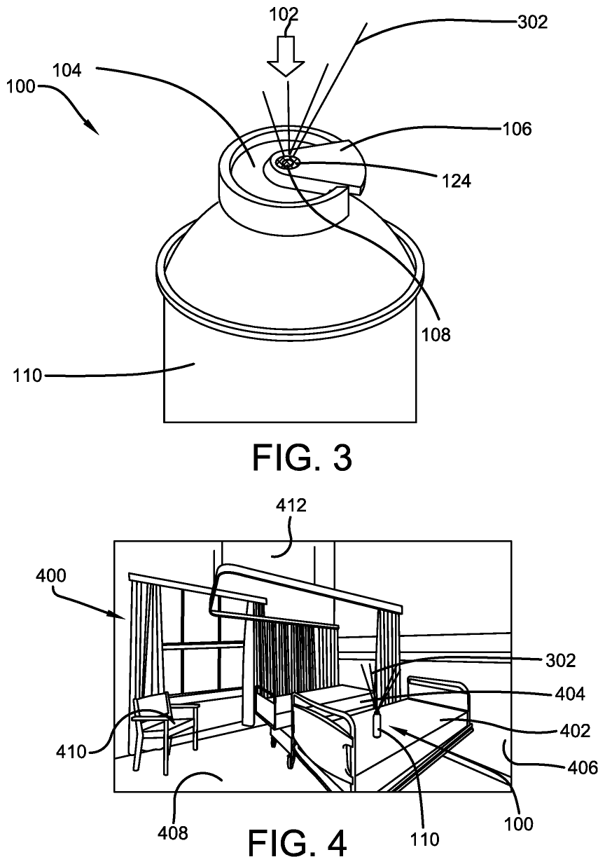

Referring initially to the drawings, FIG. 1 illustrates a perspective view of one potential embodiment of the disinfecting fogger spray device 100 of the present invention showing the cylinder 110 and the nozzle components in accordance with the disclosed architecture. More specifically, the portable disinfecting fogger device 100 comprises a pressure-activated cylinder 110 having a base 1101 and a collar 118, which may be threaded or provided with a snap ring to secure a cap. The cylinder 110 contains a disinfectant material or solution 112 under a higher pressure than the ambient pressure or the room pressure. The fogger device 100 also has a nozzle 104 having a movable flap 106, and a spray opening 108 positioned on the top of the nozzle 104, as best shown in FIGS. 1 and 3. The nozzle 104 is movable from a first closed position to a second open position, where it is in fluid communication with the interior of the cylinder 110 and the disinfectant solution 112 contained therein. More specifically, the nozzle 104 is slidably coupled to the cylinder 110, and is positionable in an open position by pressing it in a downward direction 102 towards the cylinder 110, thereby facilitating the release of the already pressurized disinfectant solution 112 from the interior of the cylinder 110 through the nozzle opening/orifice 108 and into a room or other enclosed space. In this manner, the disinfectant solution 112 is distributed into and around the room by the cascading of disinfectant solution droplets or mist to disinfect the room.

By way of background and as illustrated infra, the room may be any room that has been exposed to an infectious disease, such as a hospital ward, business area, classroom in a school, hotel room, home, office or the like. Additionally, the fogger disinfectant device 100 of the present invention functions somewhat like a conventional aerosol spray can up to the point of the spray function. For this reason, some of the structure relating to conventional aerosol sprays, such as the nozzle assembly, is not shown in greater detail though it is understood to contribute to the functionality of the present invention. The cylinder 110 may also include a sensor or timer 122 for activating the device 100. In addition, the nozzle 104 may also include a screen or mesh element 124 to help control the droplet size and prevent clogging of the nozzle 104.

FIG. 2 illustrates a diagrammatic representation of one potential embodiment of the disinfectant solution 112 for use in the disinfecting fogger spray device 100 of the present invention in accordance with the disclosed architecture. More specifically, the composition of the disinfectant solution 112 relies on a category of disinfectants associated with quaternary ammonium compounds (QACs). By way of background, cleaning products that contain QACs and other disinfectants are commonly used in homes, workplaces, and public spaces. In a preferred embodiment, the disinfectant solution 112 comprises approximately 12% ammonium saccharinate as its QAC ingredient, 56% ethanol, and 32% alkyl (all percentages are based on weight). Other components such drying agents, humectants, scents, fragrances, antiseptics, sanitizers, sodium hypochlorite, essential oils, germicides, astringents, acetic acid and water, hydrogen peroxide, chlorine compounds, surfactants, and the like may also be included, and the proportionate amount of ethanol or alkyl may decrease by up to 10% to accommodate for the same, or the relevant proportions may remain the same.

Additionally, the disinfecting solution may alternatively be a sanitizing or antiseptic solution as well. For example, an alcohol based formulation of between approximately 60-80 percent isopropyl or ethyl alcohol may be the primary ingredient. The disinfecting solution may further comprise approximately between 20-40 percent or the QAC, alkyl, and other ingredients as discussed supra or infra. Other chemical disinfectants include glutaraldehyde, iodophores, ortho-phthalaldehyde, peracetic acid, and phenolics.

In other embodiments, the alkyl dimethyl benzyl ammonium saccharinate may be selected from the group consisting of C14, C12 and C16 dimethyl benzyl ammonium saccharinates. In a further refinement, the alkyl dimethyl benzyl ammonium saccharinate comprises a mixture of C14, C12 and C16 alkyl dimethyl benzyl ammonium saccharinates. In another refinement, a short chain monohydric alcohol may be used with water as a co-solvent. Isopropanol, butanol, ethanol and propanol may also be used.

Ethanol is a preferred co-solvent and enhances the solubility of the quaternary ammonium salt and the fragrances. Nonetheless, other relatively short chain, low molecular weight, mono-hydric alcohols may be substituted or combined with the ethanol. The volume of ethanol also contributes to the rate at which the disinfectant 112 evaporates, thereby leaving behind the active component of the disinfectant which will continue to have a beneficial effect on any of the surfaces with which it comes into contact.

In an alternate embodiment of the present invention, benzalkonium chloride may also be used, which is a mixture of alkylbenzyl dimethyl ammonium chlorides of various alkyl chain lengths. Benzalkonium chlorides are particularly effective against bacteria, viruses and fungi and protozoa. In addition, these compounds are considered safe for humans, and have even been included in formulations intended for ingestion. The combination of efficacious and safe effects makes for an effective selection of the benzalkonium chlorides for use in the disinfecting fogger device 100.

The propellant that is used is not included in the formulation of the disinfectant solution 112. More specifically, the propellant is inert, and does not form a part of the disinfectant solution 112. Nonetheless, the propellant needs to be effective to discharge substantially all of the contents of the cylinder 110, and this is a matter of selection based on the desires and needs of the manufacturer. It will also be appreciated that the disinfectant solution 112 may have different percentages by weight of the constituents as per the needs, desires and end objectives of the user and manufacturer. Accordingly, in an alternate embodiment, the formulation of the disinfectant solution 112 may be comprised of approximately 5-15% ammonium saccharinate, 40-70% ethanol, and 15-40% alkyl.

FIG. 3 illustrates a top partial and perspective view of one potential embodiment of the nozzle 104 of the portable disinfectant fogger device 100 of the present invention in an activated state in accordance with the disclosed architecture. More specifically, to release the disinfectant solution 112 from the interion or the cylinder 110 and distribute the same comprehensively and evenly throughout a room, the nozzle 104 is pressed in a downward direction 102 towards the cylinder 110 by operation of the flap 106 of the nozzle 104, as best shown in FIG. 3. When the flap 106 is pressed downwards, the nozzle 104 is pushed downwards and the disinfectant 112 is released as a mist or spray 302 through the opening 108, thereby allowing the disinfectant 112 to escape in a very controlled manner. After use, the nozzle 120 is manually or automatically released (e.g., because of the lack of further internal pressure in cylinder 100).

FIG. 4 illustrates a perspective view of one potential embodiment of the disinfectant fogger device 100 of the present invention in an active state in an examining room 400 of a hospital in accordance with the disclosed architecture. More specifically, the hospital room 400 is typically comprised of a bed 402, table 404, walls 406, floor 408 and a chair. The disinfectant fogger device 100 is preferably placed in a location that is substantially near the center of the room 400, without its cap 120, and at a height calculated to allow the spray 302 that is ejected therefrom to be able to fall onto or contact all or substantially all of the surfaces in the room 400. In this manner, the disinfectant solution 112 contacts as many of the surfaces, walls 406 and ceiling, including the furniture and other things in the room 400, as possible, thereby disinfecting and sanitizing the room 400 without requiring one or more individuals to manually disinfect the same, which is time consuming, expensive and less effective.

The room 400 is preferably left closed off for a selected amount of time to allow the disinfectant solution 112 to fully disinfect the room 400, and for the solution to dry. The person activating the fogger can then exit the room 400, and close the door to confine the dispersing spray 302 to the closed room 400. The spray 302 is propelled outwardly and upwardly to a height near or at the ceiling 412 of the room 400. The spray droplets 302 then fall downward and impinge on succeeding upwardly propelled droplets. The net effect is a mushrooming of the spray droplets 302 throughout the entire room 400, descending to contact all exposed surfaces. As discussed supra, a dwell time of approximately between 30 seconds and two minutes is optimal for many indoor residential applications. However, formulations adapted for a dwell time of at least ten minutes may also be used, especially in health care or higher risk residential applications.

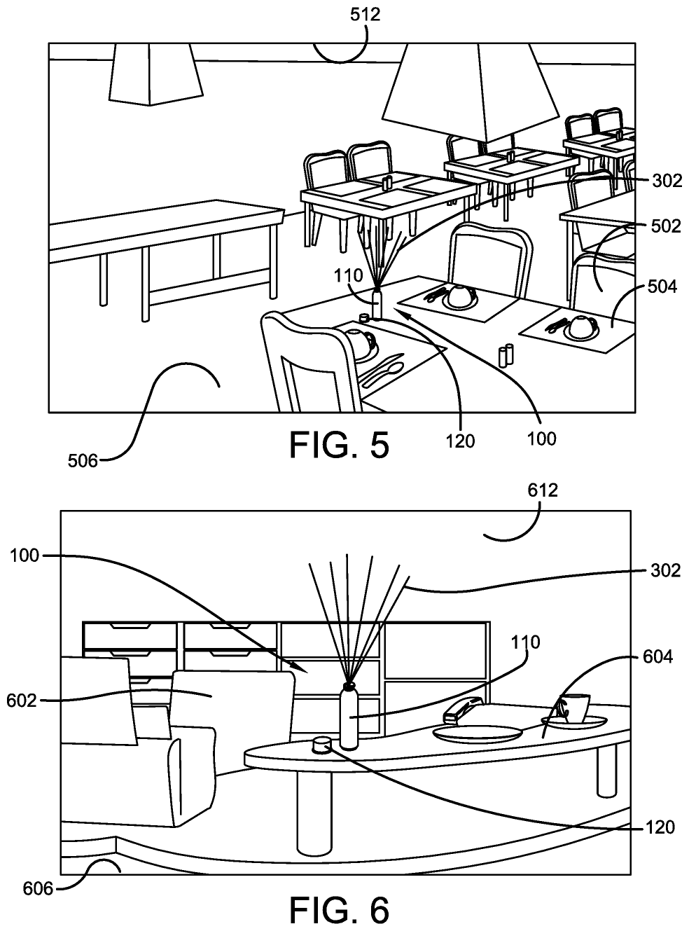

Similarly, FIG. 5 illustrates a perspective view of one potential embodiment of the disinfectant fogger device 100 of the present invention in an active state in a restaurant area 500 in accordance with the disclosed architecture. In the restaurant area 500 there is typically a chair 502, table 504; floor 506, and ceiling 512, as would be the case for virtually any restaurant 500 of this type. The disinfectant fogger device 100 is preferably placed appropriately for use with an open cap 120 and in a location that is substantially near the center of the restaurant area 500 to be disinfected, and at a height calculated to allow the spray 302 that is ejected therefrom to be able to fall onto or contact all the surfaces in the area of the restaurant that is being treated. In this manner, the disinfectant solution 112 contacts all surfaces, walls, floor 506, ceiling 512, including the furniture 502, thereby disinfecting and sanitizing the restaurant area 500 without requiring one or more individuals to manually disinfect the restaurant area 500. The spray 302 is propelled outwardly and upwardly to a height near or at the ceiling 512 of the restaurant area 500. The spray droplets then fall downward and impinge on succeeding upwardly propelled droplets. The net effect is a mushrooming of the spray droplets 302 throughout the entire room 500, descending to contact all exposed surfaces.

As yet another example of the usefulness of the present invention, FIG. 6 illustrates a perspective view of one potential embodiment of the disinfectant fogger device 100 in an active state in an office environment or area 600 in accordance with the disclosed architecture. More specifically, most office areas 600 typically comprise one or more chairs 602, tables 604, walls 612; and floor 606, etc. The disinfectant fogger device 100 is placed appropriately for use with an open cap 120 and in a location that is substantially near the center of the office space 600, and at a height calculated to allow the spray 302 that is ejected to be able to fall onto or contact all a the surfaces in the office space 600. In this manner, the disinfectant solution 112 contacts substantially all the surfaces, walls 612 and ceiling, including the furniture 602, 604 and other things in the office space 600, thereby disinfecting the office space 600 without requiring one or more individuals to manually disinfect the same. Further, and as previously stated, the spray 302 is propelled outwardly and upwardly to a height near or at the ceiling of the office space 600. The spray droplets 302 then fall downward and impinge on succeeding upwardly propelled droplets. The net effect is a mushrooming of the spray droplets 302 throughout the entire office space 600, descending to contact all exposed surfaces.

Additionally, the disinfecting solution 112 may be electrostatically charged by the disinfectant fogger device 100. The disinfectant fogger device 100 may further comprise an electrode (not shown) positioned near the nozzle 104 within the cylinder 110. When the disinfecting solution 112 is expelled by the nozzle 104, the disinfecting solution 112 is combined with air and atomized by the electrode so that the spray 302 contains positively charged particles that are able to aggressively adhere to surfaces and objects. After the spray 302 is applied, the disinfecting solution 112 works to disinfect the covered surfaces.

A previously stated, in an alternate embodiment of the present invention, a sensor module or timer 122 may be present that may include one or more status sensors, such as motion, heat sources, and/or other sensors, that prevent the device 100 from initiating while the sensors detect activity in the room. Further, a timing controller can be programmed with a specific time duration as desired by the user to automatically release the disinfectant solution 112 into the surroundings at the appropriate time. For example, if the sensor module senses conditions that are not suitable or ideal for disinfection of the room (e.g., if a person or activity is present), the defogger device 100 may not release the disinfecting solution 112 into the room at that time.

Additionally, the cylinder 110 will be available in different sizes and colors to accommodate different user needs and preferences. Exact size, measurement, construction and design specifications of the unique fogger device 100 of the present invention may vary upon manufacturing, or the particular material that is being used. Additionally, the cylinder 110 may have a name tag, name badges, laser-graving, customizable colors and fonts, embroidery and prints.

In a preferred embodiment, the cylinder 110 may contain 8 to 20 ounces or more of the disinfectant solution 112. Further, the solution may comprise a fragrance or scent that accounts for between 0.5 and 5 percent of the solution 112. The expelled liquid droplets 302 are generally benign to all surfaces that they contact, whether made of fabric, wood, paint, paper, leather, etc. and will not stain. The solution will, however, disinfect and sanitize these surfaces. The entire dispersal of the disinfecting solution 112 is preferably accomplished in between 15 seconds and 1 minute, with the droplets drying soon thereafter. Thereafter, the room may be entered to collect and dispose of the fogger device 100 and the room may be returned to normal use, with the assurance that the exposed surfaces and air have been properly sanitized. More specifically, the disinfectant solution 112 according to the present invention kills up to 99.9 percent of all germs existing in the air space where the disinfectant 112 is emitted. The disinfecting substance 112 may be a room temperature (e.g., 20° C. to 25° C.) substance that can be dispersed as a fog or mist during operation. In other embodiments, the disinfecting substance 112 may be used at temperatures in the range of between about −40° C. to 100° C.

The advantages of the disinfecting device 100 of the present invention can be seen with respect to the ability to have a thorough sanitizing of the subject room. With the advent of the present invention, the distribution of the disinfectant solution 112 ensures a more thorough coverage than would otherwise be achieved by hand cleansing of the surfaces, and is much less time consuming and labor intensive. The fogger device 100 of the present invention may be offered as a single-use type of product where it is discarded once it has been used. In the alternate, the fogger device 100 may be collected and recycled by a manufacturer merely by removing the spray nozzle 104 (and refurbishing if necessary), refilling the cylinder 110 with more disinfecting solution 112, and re-installing the spray nozzle 104.

It should be noted that the terms spray, mist, and disperse are used as examples throughout the specification and claims, however, embodiments described as spraying may also be misting and/or dispersing. Similarly, embodiments described as misting may also be spraying and/or dispersing and embodiments described, as dispersing may also be misting and/or spraying.

Likewise, certain terms are used throughout the following description and claims to refer to particular features or components. As one skilled in the art will appreciate, different persons may refer to the same feature or component by different names. This document does not intend to distinguish between components or features that differ in name, but not structure or function. As used herein “portable disinfecting fogger product”, “disinfecting fogger”, “fogger device”, and “disinfecting fogger product” are interchangeable and refer to the portable disinfecting fogger product 100 of the present invention.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Notwithstanding the forgoing, the portable disinfecting fogger device 100 of the present invention can be of any suitable size and configuration as is known in the art without affecting the overall concept of the invention, provided that it accomplishes the above stated objectives. One of ordinary skill in the art will appreciate that the size, configuration and material of the portable disinfecting fogger device 100 as shown in the FIGS. are for illustrative purposes only, and that many other sizes of the portable disinfecting fogger device 100 are well within the scope of the present disclosure. Although the dimensions of the portable disinfecting fogger device 100 are important design parameters for user convenience, the portable disinfecting fogger device 100 may be of any size and shape that ensures optimal performance during use and/or that suits pet owners need and/or preference.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A disinfectant emitting device comprising:
a container sized and configured to receive a disinfecting solution under pressure, the container having a flat base on one end and a nozzle on a second end opposite the flat base, the nozzle being movable between a first and second position, wherein the nozzle is substantially surrounded by a collar; and
a disinfecting solution contained within the container and held at a pressure higher than an ambient pressure outside the container, wherein the disinfecting solution including between 5-15% of a quaternary ammonium compound, between about 40-70% of a monohydric alcohol, and between about 15-40% of an alkyl, all by weight.

2. The disinfecting emitting device of claim 1, wherein the quaternary ammonium compounds is ammonium saccharinate.

3. The disinfecting emitting device of claim 1, wherein the monohydric alcohol is one of an isopropanol, a butanol, an ethanol and a propanol.

4. The disinfecting emitting device of claim 1, wherein the monohydric alcohol is ethanol.

5. The disinfecting emitting device of claim 1, wherein the disinfecting solution further comprises a benzalkonium chloride.

6. The disinfecting emitting device of claim 1, wherein the disinfecting solution includes a drying agent selected from the group of a calcium sulfate, a sodium sulfate, a calcium chloride and a magnesium sulfate, and further wherein the drying agent ranged from between 0.5 and 15% of the disinfecting solution by weight.

7. The disinfecting emitting device of claim 1, wherein the container further comprises one of a sensor or a timer for remote activation of the disinfectant emitting device.

8. The disinfecting emitting device of claim 1, wherein the disinfecting solution upon dispersal from the container has a droplet diameter size ranging from about 5 microns to about 50 microns.

9. The disinfecting emitting device of claim 1, wherein the disinfecting solution upon dispersal from the container has a droplet diameter size ranging from about 10 microns to about 35 microns.

10. The disinfecting emitting device of claim 1, wherein the disinfecting solution has a predefined dispensing rate, and an effective solution concentration of between 10 and 90%.

11. The disinfecting emitting device of claim 1, wherein the disinfecting solution is comprised of 12% of ammonium saccharinate, 56% of ethanol, and 32% of alkyl, all by weight.

12. The disinfecting emitting device of claim 1, wherein the disinfecting solution is comprised of a fragrance of between 0.5 and 5% by weight.

13. The disinfecting emitting device of claim 1, wherein the container further comprises a screen disposed below the nozzle for controlling a droplet size of the disinfecting solution.

14. A disinfecting and sanitizing fogger device comprising:
a container for holding a content under a pressure that is greater than an ambient pressure outside of the container, wherein the container is further comprised of a flat bottom, a nozzle, at least one of a sensor or a timer to trigger activation of the container, and a screen disposed adjacent to the nozzle;
the nozzle having a first position and a second position; and
a disinfecting solution held within the container at the pressure, wherein the disinfecting solution comprises between 5-15% of an ammonium saccharinate, between 40-70% of a monohydric alcohol, and between 15-40% of an alkyl, all by weight, and further wherein the screen ensures that the disinfecting solution is dispersed from the container having a droplet diameter size of between 5 and 50 microns.

15. The disinfecting and sanitizing fogger device of claim 14, wherein the disinfecting solution has a drying time of less than 60 seconds after being dispersed from the container.

16. The disinfecting and sanitizing fogger device of claim 14, wherein the disinfecting solution comprises a fragrance of between 0.5 and 5% of the disinfecting solution by weight.

17. The disinfecting and sanitizing fogger device of claim 14, wherein the screen ensures that the disinfecting solution is dispersed from the container having a droplet diameter size of between 10 and 35 microns.

18. The disinfecting and sanitizing fogger device of claim 14, wherein the disinfecting solution is only dispersed from the container when the nozzle is in the second position.

19. A method of using a disinfecting and sanitizing fogger device comprising the steps of:
selecting an area to be disinfected and sanitized;
providing a canister having a pressurized disinfecting solution contained therein, a flat bottom, a nozzle, and one of a sensor or a timer for remotely activating the canister, wherein the nozzle is repositionable between a first closed position and a second opened position and the canister further comprises a screen positioned adjacent to the nozzle;
filling and pressurizing the canister with a disinfecting and sanitizing solution comprised of between 5-15% of an ammonium saccharinate by weight, between 40-70% of a monohydric alcohol by weight, and between 15-40% of an alkyl by weight;
setting one of the timer or the sensor; and
moving the nozzle from the first position to the second position to release the disinfecting and sanitizing solution upon a triggering of the timer or the sensor, wherein the screen ensures that the disinfecting and sanitizing solution is dispersed from the canister having a droplet diameter size of between 10 and 35 microns.

20. The method of using a disinfecting and sanitizing fogger of claim 19, wherein the monohydric alcohol is one of an isopropanol, a butanol, an ethanol and a propanol.
